(19) United States
(12) Patent Application Publication
(10) Pub. No.: US 2012/0115173 A1
(43) Pub. Date: May 10, 2012

(54) BIOMARKERS FOR MOTOR NEURON DISEASE

(75) Inventor: Robert P. Bowser, Scottsdale, AZ (US)

(73) Assignee: University of Pittsburgh - Of the Commonwealth System of Higher Education, Pittsburgh, PA (US)

(21) Appl. No.: 13/350,305

(22) Filed: Jan. 13, 2012

Related U.S. Application Data
(63) Continuation of application No. 12/209,899, filed on Sep. 12, 2008.
(60) Provisional application No. 60/971,709, filed on Sep. 12, 2007.

(51) Int. Cl. G01N 33/566; C12Q 1/37

(52) U.S. Cl. 435/7.92; 435/7.1; 435/23

(57) ABSTRACT

The invention provides methods of determining a diagnosis or prognosis of motor neuron disease in a mammal comprising determining the expression level of one or more proteins or polypeptides of the renin-angiotensin system in a sample taken from a subject. Similarly, aberrant post-translational modification of the proteins or polypeptides as compared to a negative control indicates a diagnosis of disease.

### CROSS-REFERENCE TO RELATED APPLICATIONS

This patent application is a continuation of copending U.S. patent application Ser. No. 12/209,899, filed Sep. 12, 2008, which claims priority to U.S. Provisional Patent Application 60/971,709, filed Sep. 12, 2007, the entire contents of each of which are incorporated herein by reference.

### STATEMENT REGARDING FEDERALLY SPONSORED RESEARCH AND DEVELOPMENT

This invention was made with Government support under Grant Number ES 013469 awarded by the National Institutes of Health. The Government has certain rights in this invention.

### BACKGROUND OF THE INVENTION

Motor neuron disease is a family of disorders characterized by progressive degeneration of upper and/or lower motor neurons. The most common form of adult-onset motor neuron disease is Amyotrophic Lateral Sclerosis (ALS), also known as Lou Gehrig's disease. Other forms of motor neuron disease include primary lateral sclerosis, progressive muscular atrophy, pseudobulbar palsy, progressive bulbar palsy, lower motor neuron disease and spinal muscular atrophy.

There currently is one FDA approved drug for ALS (rilutek), but this only slows progression by a few months. Rapid diagnosis could potentially improve drug effects by introducing drug earlier in the disease course. However, diagnosis of motor neuron diseases such as ALS has been made clinically through neurological examination and exclusion of other disorders having similar manifestations. Diagnosis typically takes 6-12 months and multiple visits to specialists. Rapid diagnostic tests are not currently available for ALS and no markers of disease progression have been previously identified.

There remains a need for improved methods for identifying therapeutic targets of motor neuron disease, especially ALS, and improved methods of diagnosing the disease. Biomarkers of motor neuron disease could potentially assist measurements of drug efficacy in clinical trials and identify novel therapeutic targets for the generation of improved drug therapies.

### BRIEF SUMMARY OF THE INVENTION

The invention provides methods of determining a diagnosis or prognosis of motor neuron disease in a mammal comprising determining the expression of one or more proteins of the renin-angiotensin system in a sample taken from a subject (e.g., a patient). Proteins in the pathway (or polypeptides from such proteins) can be quantified and compared to a negative control, wherein an aberrant quantity of one or more of the proteins or polypeptides indicates a diagnosis of motor neuron disease. Specifically, an increased quantity of certain proteins or polypeptides associated with the renin-angiotensin pathway indicates a diagnosis of disease, while a decreased quantity of certain proteins or polypeptides associated with the renin-angiotensin pathway indicates a diagnosis of disease. Similarly, aberrant post-translational modification of the proteins or polypeptides as compared to a negative control indicates a diagnosis of disease.

### BRIEF DESCRIPTION OF THE SEVERAL VIEWS OF THE DRAWING(S)

FIG. 1 compares base peak chromatograms of an ALS sample and control sample generated from a full MS scan.

FIG. 2 presents exemplary base peak chromatograms of an ALS sample.

FIG. 3 presents exemplary base peak chromatograms of a control sample.

### DETAILED DESCRIPTION OF THE INVENTION

In one aspect, the invention provides a method of diagnosing of motor neuron disease in a mammal. In one embodiment, the method comprises (1) providing a sample taken from a patient; (2) measuring or quantifying in the sample the amount of one or more proteins or polypeptides in the renin-angiotensin system (e.g., polypeptides comprising any of SEQ ID NOs: 1-63, polypeptides comprising a majority of contiguous amino acids of any of SEQ ID NOs: 1-63, or proteins associated therewith (see Table 1)); and (3) comparing the amount of the one or more protein or polypeptide sequences with a negative control. In accordance with this aspect of the invention, the presence of an aberrant quantity of one or more proteins or polypeptide sequences compared to the negative control indicates a diagnosis of motor neuron disease.

In some embodiments, one or more of the measured proteins or polypeptides is a protein or polypeptide for which increased levels relative to a negative control are associated with a diagnosis of motor neuron disease (e.g., SEQ ID NOs: 1-42, a polypeptide comprising a majority of contiguous amino acids from SEQ ID NOs: 1-42, or the proteins associated therewith (see, e.g., Table 1)). In other embodiments, one or more of the measured proteins or polypeptides is a protein or polypeptide for which decreased levels relative to a control are associated with a diagnosis of motor neuron disease (e.g., SEQ ID NOs: 43-63, a polypeptide comprising a majority of contiguous amino acids of SEQ ID NOs: 43-63, or a protein associated therewith (see Table 1)). In a preferred embodiment, two or more proteins or polypeptides are measured, with at least the first comprising a majority of contiguous amino acids of one of SEQ ID NOs: 1-42 and at least the second comprising a majority of contiguous amino acids of one of SEQ ID NOs: 43-63. For example the first can consist of or consist essentially of one of SEQ ID NOs: 1-42 and the second can consist of or consist essentially of one of SEQ ID NOs: 43-63.

In some embodiments, the diagnostic method further comprises detecting aberrant post-translational modification of the protein or polypeptide sequence(s) as compared to the control sample, wherein the presence of aberrant post-translational modification indicates a diagnosis of motor neuron disease. Thus, for example, the invention provides a method of diagnosing motor neuron disease in a mammal comprising (1) providing a sample taken from a patient; (2) evaluating the sample for post-translational modification of one or more peptides of proteins in the renin-angiotensin pathway (e.g., polypeptides comprising any of SEQ ID NOs: 1-63, polypeptides comprising a majority of contiguous amino acids of any of SEQ ID NOs: 1-63, or proteins associated therewith (see Table 1)); and (3) comparing the post-translational modification of the peptides with a negative control. Aberrant post-translational modification of the peptides as compared to a negative control indicates a diagnosis of disease.

The post-translational modification can be any modification detectable by any method known to one of ordinary skill in the art, such as mass spectrometry proteomics. For example, the post-translational modification can be phosphorylation, glycosylation, oxidation, or methylation. Exemplary protocols for determining levels of post-translational modifications are provided in Chi et al., Proc Natl Acad Sci, USA 104(7): 2193-2198 (2007); Burlingame, et al., Methods 36: 383-394 (2005); Webb D J, et al., J Cell Sci 118: 4925-4929 (2005).

The negative control for the diagnostic methods can be any suitable negative control known to one of ordinary skill in the art. In a preferred embodiment, the negative control is a sample taken from a non-diseased subject of the same species as the patient, i.e., a healthy subject. In other embodiments, the negative control is a profile of measurements understood to reflect expected levels of the peptides a non-diseased subject. In some embodiments, additional controls can be also be compared to the test sample, such as samples taken from diseased subjects or profiles of measurements understood to reflect peptide levels associated with motor neuron disease.

Without being bound by any particular theory, it is thought that SEQ ID NOs: 1-63 represent biomarkers for proteins relating to the renin-angiotensin system. Such proteins can be within the renin-angiotensin pathway, i.e., respond to renin-angiotensin activation, or can modulate the renin-angiotensin pathway. For example, the renin-angiotensin pathway is thought to be modulated by related systems, such as the plasma kallikrein/kinin system. See, e.g., Schmaier, Am. J. Physiol. Regul. Integr. Comp. Physiol. 285:1-13 (2003). In the present invention, SEQ ID NOs: 1-63 are thought to represent one or more proteins such as angiotensin precursor, kallikrein 6 (isoform B and isoform A preproprotein), kininogen, clusterin, antihrombin III (Serpin C1), plasminogen, plasminogen activator/urokinase, pigment epithelial derived factor precursor (PEDF or Serpin F1), vitamin D binding protein precursor, Vitamin D binding protein variant, angiotensin preproprotein, insulin-like growth factor binding protein 6, insulin-like growth factor binding protein 7, coagulation factor II precursor, coagulation factor XII, or plasminogen activator (urokinase receptor isoform).

Exemplary biomarker sequences are provided in Table 1, indicating the protein represented by the polypeptide sequence. Periods (“.”) are putative sites for trypsin cleavage.

TABLE 1

| | | | |
|---|---|---|---|
| | Increased/ | | Increased/ |
| | Decreased | | Decreased |
| | in ALS | | in ALS |
| Sequence | (+/−) | Sequence | (+/−) |

Protein: Angiotensin precursor (SwissProt Accession number P01019)

| | | | |
|---|---|---|---|
| R.FMQAVTGWK.T | + | K.ANAGKPKDPTFIPAPIQAK.T | − |
| (SEQ ID NO: 1) | | (SEQ ID NO: 43) | |
| K.ALQDQLVLVAAK.L | + | | |
| (SEQ ID NO: 2) | | | |
| R.SLDFTELDVAAEK.I | + | | |
| (SEQ ID NO: 3) | | | |
| R.LQAILGVPWK.D | + | | |
| (SEQ ID NO: 4) | | | |

Protein: Angiotensin precursor (SwissProt Accession number P01019)(continued)

| | |
|---|---|
| K.QPFVQGLALYTPVVLPR.S | + |
| (SEQ ID NO: 5) | |
| K.VLSALQAVQGGLLVAQGR.A | + |
| (SEQ ID NO: 6) | |

Protein: Kallikrein 6 (SwissProt Accession number Q92876)

| | | | |
|---|---|---|---|
| K.DSCQGDSGGPLVCGDHLR.G | + | R.LARPAKLSELIQPLPLER.D | − |
| (SEQ ID NO: 7) | | (SEQ ID NO: 44) | |
| K.TADGDFPDTIQCAYIHLVSR.E | + | K.LSELIQPLPLER.D | − |
| (SEQ ID NO: 8) | | (SEQ ID NO: 45) | |
| | | R.GLVSWGNIPCGSK.E | − |
| | | (SEQ ID NO: 46) | |
| | | R.QRESSQEQSSVVR.A | − |
| | | (SEQ ID NO: 47) | |
| | | K.YGKDSCQGDSGGPLVCGDHLR.G | − |
| | | (SEQ ID NO: 48) | |
| | | R.AVIHPDYDAASHDQDIMLLR.L | − |
| | | (SEQ ID NO: 49) | |

Protein: Kininogen-1 (SwissProt Accession number P01042)

| | | | |
|---|---|---|---|
| R.ETTCSKESNEELTESCETK.K | + | K.RPPGFSPFR.S | − |
| (SEQ ID NO: 9) | | (SEQ ID NO: 50) | |
| | | R.IGEIKEETTSHLR.S | − |
| | | (SEQ ID NO: 51) | |
| | | R.KLGQSLDCNAEVYVVPWEKK.I | − |
| | | (SEQ ID NO: 52) | |
| | | K.GRPPKAGAEPASER.E | − |
| | | (SEQ ID NO: 53) | |
| | | K.AATGECTATVGKR.S | − |
| | | (SEQ ID NO: 54) | |

Protein: Clusterin (activated by AT-1 receptor binding to ANG-II)
(SwissProt Accession number P10909)

| | | | |
|---|---|---|---|
| R.ELDESLQVAER.L | + | R.KTLLSNLEEAK.K | − |
| (SEQ ID NO: 10) | | (SEQ ID NO: 55) | |
| | | K.YVNKEIQNAVNGVK.Q | − |
| | | (SEQ ID NO: 56) | |

Protein: Antithrombin III (SwissProt Accession number P01008)

| | |
|---|---|
| K.LQPLDFKENAEQSR.A | + |
| (SEQ ID NO: 11) | |
| K.ATEDEGSEQKIPEATNRR.V | + |
| (SEQ ID NO: 12) | |
| K.SKLPGIVAEGR.D | + |
| (SEQ ID NO: 13) | |
| R.DDLYVSDAFHK.A | + |
| (SEQ ID NO: 14) | |
| K.TSDQIHFFFAK.L | + |
| (SEQ ID NO: 15) | |
| R.VAEGTQVLELPFK.G | + |
| (SEQ ID NO: 16) | |
| R.EVPLNTIIFMGR.V | + |
| (SEQ ID NO: 17) | |
| K.NDNDNIFLSPLSISTAFAMTK.L | + |
| (SEQ ID NO: 18) | |
| R.ITDVIPSEAINELTVLVLVNTIYFK.G | + |
| (SEQ ID NO: 19) | |

Protein: Plasminogen (SwissProt Accession number P00747)

| | |
|---|---|
| K.RAPWCHTTNSQVR.W | + |
| (SEQ ID NO: 20) | |
| K.NYCRNPDGDVGGPWCYTTNPR.K | + |
| (SEQ ID NO: 21) | |

Protein: Plasminogen activator, urokinase
(SwissProt Accession number Q03405)

| | |
|---|---|
| R.GCATASMCQHAHLGDAFSMN | + |
| HIDVSCCTK.S (SEQ ID NO: 22) | |

Protein: Pigment epithelial derived factor precursor
(PEDF or Serpin F1) (SwissProt Accession number P36955)

| | | | |
|---|---|---|---|
| K.TVQAVLTVPK.L | + | R.DTDTGALLFIGK.I | − |
| (SEQ ID NO: 23) | | (SEQ ID NO: 57) | |
| K.LQSLFDSPDFSK.I | + | | |
| (SEQ ID NO: 24) | | | |
| R.YGLDSDLSCK.I | + | | |
| (SEQ ID NO: 25) | | | |
| R.KTSLEDFYLDEER.T | + | | |
| (SEQ ID NO: 26) | | | |
| K.TSLEDFYLDEER.T | + | | |
| (SEQ ID NO: 27) | | | |
| R.ALYYDLISSPDIHGTYK.E | + | | |
| (SEQ ID NO: 28) | | | |
| R.LDLQEINNWVQAQMK.G | + | | |
| (SEQ ID NO: 29) | | | |
| K.EIPDEISILLLGVAHFK.G | + | | |
| (SEQ ID NO: 30) | | | |

Protein: Vitamin D-binding protein precursor
(SwissProt Accession number P02774)

| | | | |
|---|---|---|---|
| R.THLPEVKLSK.V | + | K.LPDATPTELAK.L | − |
| (SEQ ID NO: 31) | | (SEQ ID NO: 58) | |
| K.ELPEHTVKLCDNLSTK.N | | K.ELSSFIDKGQELCADYSENTFTEYK.K | − |
| (SEQ ID NO: 32) | | (SEQ ID NO: 59) | |
| | | K.SLGECCDVEDSTTCFNAKGPLLKK.E | − |
| | | (SEQ ID NO: 60) | |

Protein: Vitamin D-binding protein variant
(SwissProt Accession number Q53F31)

| | |
|---|---|
| K.AKLPEATPTELAK.L | + |
| (SEQ ID NO: 33) | |

Protein: Insulin−like growth factor binding protein 7 (IGFBP-7)
(SwissProt Accession number Q16270)

| | | | |
|---|---|---|---|
| R.TELLPGDRDNLAIQTR.G | + | R.GGPEKHEVTGWVLVSPLSK.E | − |
| (SEQ ID NO: 34) | | (SEQ ID NO: 61) | |
| K.EDAGEYECHASNSQGQASASAK.I | + | R.GKAGAAAGGPGVSGVCVCK.S | − |
| (SEQ ID NO: 35) | | (SEQ ID NO: 62) | |
| | | R.ITVVDALHEIPVKKGEGAEL. | − |
| | | (SEQ ID NO: 63) | |

Protein: Insulin-like growth factor binding protein 6 (IGFBP-6)
(SwissProt Accession number P24592)

| | |
|---|---|
| R.HLDSVLQQLQTEVYR.G | + |
| (SEQ ID NO: 36) | |
| R.CLPARAPAVAEENPK.E | + |
| (SEQ ID NO: 37) | |
| R.EGQECGVYTPNCAPGLQCHPPK.D | + |
| (SEQ ID NO: 38) | |

Protein: Coagulation Factor XII (SwissProt Accession number P00748)

| | |
|---|---|
| R.TTLSGAPCQPWASEATYR.N | + |
| (SEQ ID NO: 39) | |
| R.LHEAFSPVSYQHDLALLR.L | + |
| (SEQ ID NO: 40) | |
| R.NKPGVYTDVAYYLAWIR.E | + |
| (SEQ ID NO: 41) | |
| R.LCHCPVGYTGPFCDVDTK.A | + |
| (SEQ ID NO: 42) | |

It will be understood that SEQ ID NOs: 1-63 represent polypeptides obtained from trypsin digestion. In performing the inventive method, alternative proteolytic enzymes can be employed (which are known to those of ordinary skill in the art), many of which cleave between amino acids differently than trypsin. Accordingly, the inventive method can employ alternative sequences comprising a majority of contiguous amino acids from any of SEQ ID NOs: 1-63, but which can also comprise additional amino acids at either or both ends. Moreover, it will be observed that a polypeptide resulting from trypsin cleavage can lack the amino and carboxy terminal amino acids of SEQ ID NOs:1-63 (left and right of the “.” in Table 1), inasmuch as these are cleaved and not present in the digested polypeptide. It should be noted, however, that SEQ ID NO:63 represents the carboxy-terminus of the mature protein, thus the polypeptide is expected to possess the terminal leucine residue.

In another aspect, the invention provides a method of determining a prognosis of a motor neuron disease by assessing a changed level of one or more proteins or polypeptides of the rennin-angiotensin pathway relative to a control. In one embodiment, the prognostic method comprises (1) providing a sample taken from a patient previously diagnosed with a motor neuron disease; (2) measuring or quantifying in the sample the amount of one or more proteins or polypeptides in the renin-angiotensin pathway, such as SEQ ID NOs: 1-42, polypeptides comprising a majority of contiguous amino acids of SEQ ID NOs: 1-42, or the proteins associated therewith (see Table 1); and (3) comparing the amount of the one or more protein or polypeptide sequences with a control. In this aspect of the invention, the presence of an increased quantity of the one or more protein or polypeptide sequences compared to the control indicates a prognosis of advancing disease, and a decreased or unchanged quantity compared to the control indicates a prognosis of remission or non-advancing disease as determined by clinical parameters.

In another embodiment, the prognostic method comprises (1) providing a sample taken from a patient previously diagnosed with a motor neuron disease; (2) measuring or quantifying in the sample the amount of one or more proteins or polypeptides in the renin-angiotensin pathway, such as SEQ ID NOs: 43-63, polypeptides comprising a majority of contiguous amino acids of SEQ ID NOs: 43-63, or proteins associated therewith; and (3) comparing the amount of the one or more protein or polypeptide sequences with a control. In this aspect of the invention, the presence of a decreased quantity of the one or more protein or polypeptide sequences compared to the control indicates a prognosis of advancing disease, and an increased or unchanged quantity compared to the control indicates a prognosis of remission or non-advancing disease as determined by clinical parameters.

In some embodiments, the prognostic method further comprises detecting aberrant post-translational modification of the one or more peptide sequences as compared to the control sample, wherein the presence of aberrant post-translational modification indicates a diagnosis of motor neuron disease. Thus, for example, the invention provides a method of determining a prognosis of a motor neuron disease in a mammal comprising (1) providing a sample taken from a patient; (2) evaluating the sample for post-translational modification of one or more proteins or polypeptides in the renin-angiotensin pathway, (e.g., polypeptides comprising any of SEQ ID NOs: 1-63 or a majority of contiguous amino acids from SEQ ID NOs:1-63, and the proteins associated therewith (see Table 1)); and (3) comparing the post-translational modification of the proteins or polypeptides with a control. It will be observed that, according to this aspect of the invention, increased aberrant post-translational modification of the proteins or polypeptides as compared to the control indicates a prognosis of advancing disease, and decreased level of aberrant post-translational modification of the proteins or polypeptides as compared to the control indicates a prognosis of remission or non-advancing disease as determined by clinical parameters.

The control for use in the prognostic method can be, for example, a prior sample from the patient and/or a pre-determined expression profile. In other embodiments, the control is a profile of measurements understood to reflect expected levels of the peptides a subject, which can be a non-diseased subject of the same species as the patient or one with a disease at an early or advanced stage relative to the patient. In some embodiments, additional controls can be also be compared to the test sample, such as samples taken from diseased subjects or profiles of measurements understood to reflect peptide levels associated with motor neuron disease.

In any aspect of the invention, the amount of the peptide sequence(s) can be measured using any method known to one of ordinary skill in the art, such as mass spectrometry, ELISA, or Western blot. In a preferred embodiment, mass spectrometry is used. In a more preferred embodiment, the mass spectrometry is liquid chromatography mass spectrometry/mass spectrometry (LC-MS/MS). Exemplary protocols for performing LC MS/MS to quantify peptides are provided in Nagele, et al., Exp Rev Proteomics 1(1): 37-46 (2004); Peng J, et al., J Proteome Res 2(1): 43-50 (2003); and Qian W J, et al., J Proteome Res 4(1): 53-62 (2005). Exemplary mass spectrometry methods are also described in US Pub. No. 2005/0148026, the contents of which are incorporated herein by reference. Additional mass spectrometry based methodologies to identify peptide and protein alterations include MALDI-MS/MS (see, e.g., Pan, et al., Anal. Chem. 75: 1316-1324 (2003).

One of ordinary skill in the art can use any suitable statistical calculation for determining whether concentration levels of a protein or polypeptide, or levels of post-translational modification thereof, are increased or decreased relative to a control. It will be understood that levels are significantly different from control if the test sample differs from the control sample by more than 1%. In some embodiments, peptide levels may differ from the control sample by more than 5%. In other embodiments, peptide levels may differ from the control sample by 10%, 20%, 30%, 40%, 50%, 60%, 70%, 80%, 90%, 100%, 200%, 500%, 1000% or more, as well as intervening values.

In performing the inventive methods, the sample can be any suitable tissue sample such as cerebrospinal fluid, blood, or urine. In some preferred embodiments, the sample is cerebrospinal fluid. The sample can be obtained by any method known to one of ordinary skill in the art.

The motor neuron disease can be any motor neuron disease, such as amyotrophic lateral sclerosis (ALS), primary lateral sclerosis, progressive muscular atrophy, pseudobulbar palsy, progressive bulbar palsy, lower motor neuron disease and spinal muscular atrophy. In a preferred embodiment, the motor neuron disease is amyotrophic lateral sclerosis (ALS).

The patient or the subject from whom a control sample is obtained can be a human or any suitable non-human mammal such as a mouse, rat, rabbit, cat, dog, pig, sheep, cow, or primate. In some embodiments, the subject is a non-human experimental animal model. In a preferred embodiment, the subject a primate. In a more preferred embodiment, the subject is a human.

The following examples further illustrate the invention but, of course, should not be construed as in any way limiting its scope.

### Example 1

This example demonstrates a method of measuring peptide biomarkers of motor neuron disease.

A sample of cerebrospinal fluid (CSF) is taken from a patient diagnosed with ALS and a control sample is taken from a non-diseased control subject. The most abundant proteins are removed by affinity chromatography and the samples digested with trypsin and peptides enriched prior to liquid chromatography mass spectrometry (LC-MS/MS).

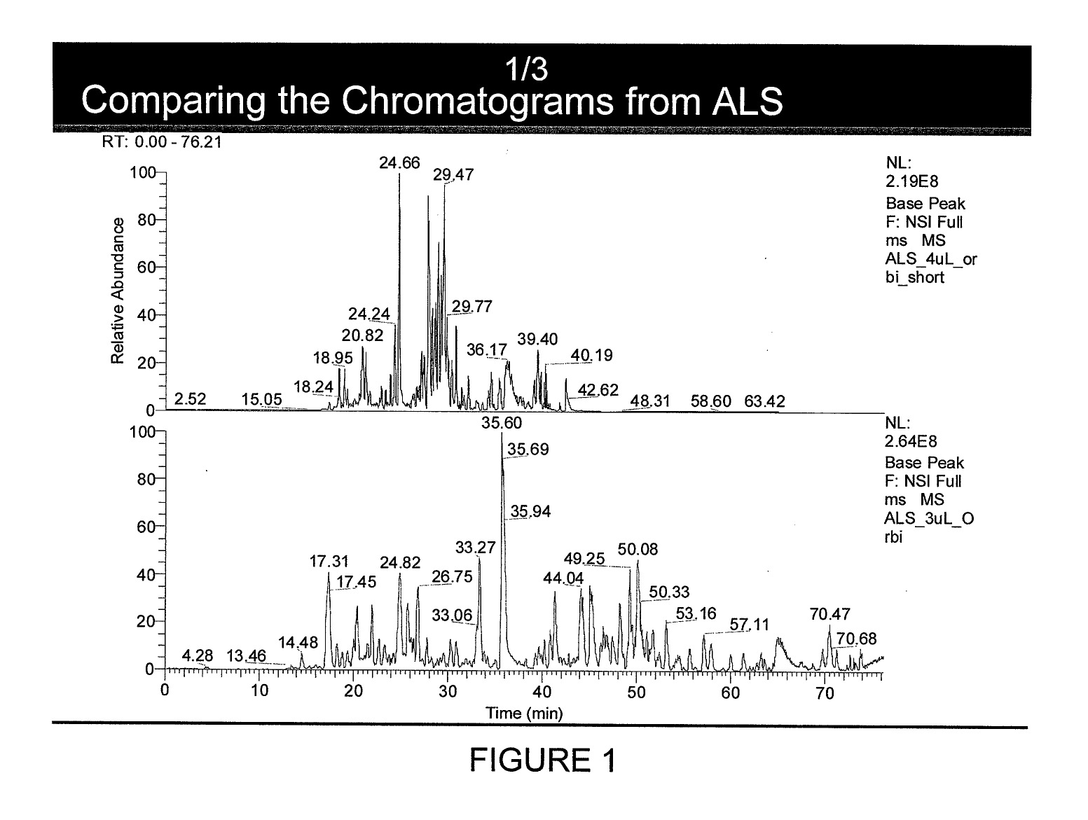

Protein first dimension liquid chromatography (1D LC) is performed on the ALS and control samples using a 34, injection into a ZORBAZ 300SB-C-18 column, 5 μm particle size, 5×0.3 mm trap, or a customized gradient (Picofrit, Proteopep 2, 5 μm particle size, 75 μm ID×15 μm tip×10 cm length) for analysis on the Thermo LTQ-Orbitrap. Using these gradients, base peak chromatograms of the ALS sample and control sample are generated from a full MS scan as shown in FIG. 1 (“Comparing the Chromatograms from ALS”). Peptide identification is performed on each peak to generate a complete peptide list, and proteins determined by SEQUEST database (Human_ref.fasta, parsed from nr.fasta) comparisons. The peptide mass window is +/−2 Da, and the peptide mass range is 600-5000 Da. Monoisotopic parent and fragment ions are identified with a minimum ion count of 10. Filter applied on the search results are HUPO recommendation: XCorr>1.9, 2.2, 3.7 for 1+, 2+, 3+ peptides, respectively RSP<5, Delta CN>0.1, with greater than or equal to 2 different peptides per protein.

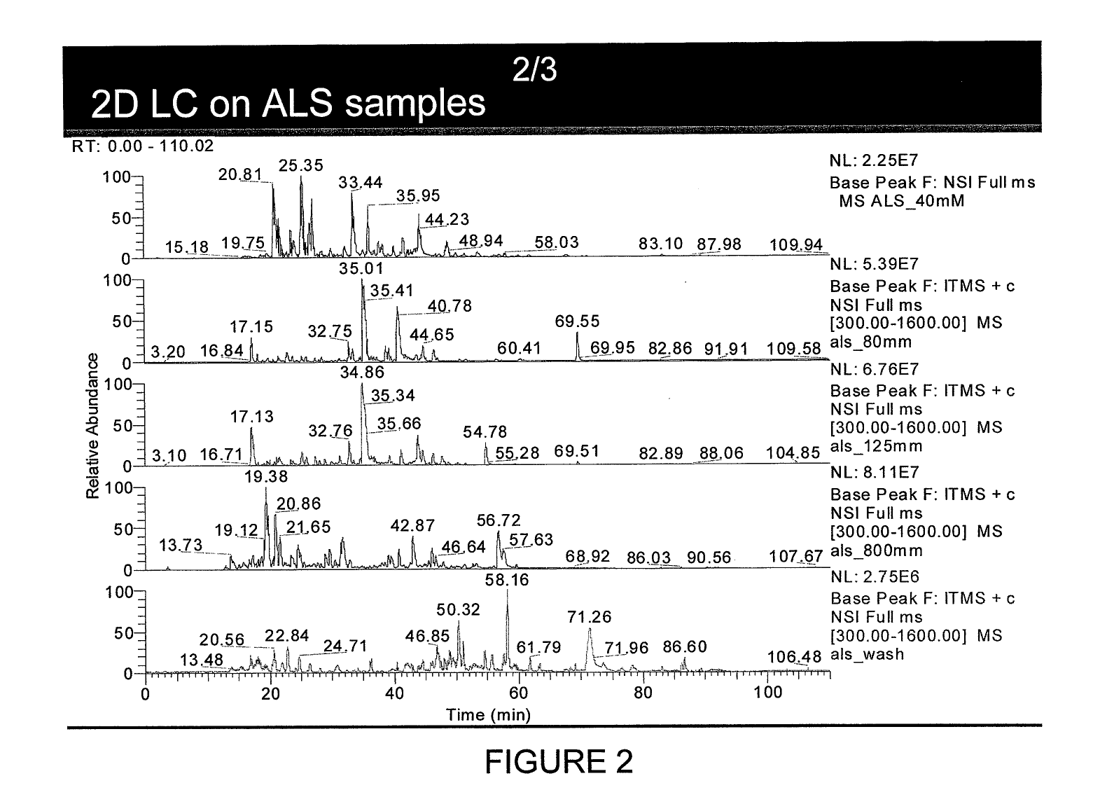

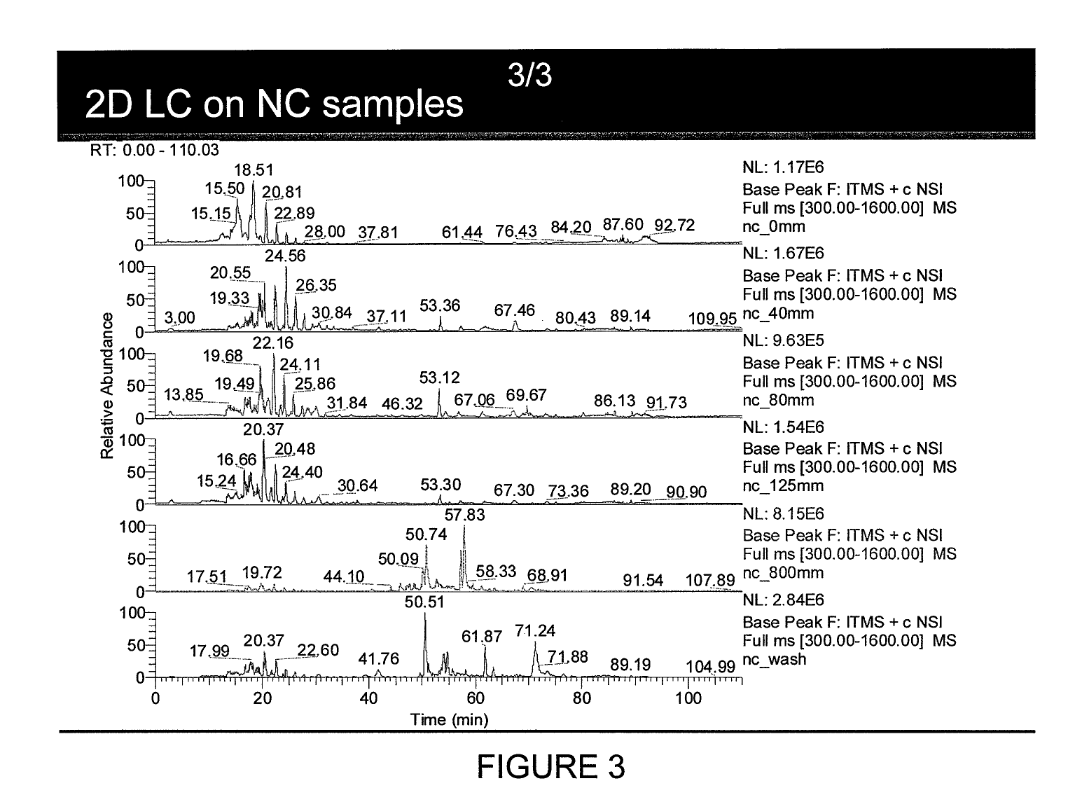

Protein second dimension liquid chromatography (2D LC) is performed for additional analysis of the proteome. For CSF, a 204 injection into a Picofrit, Thermobiobasic C18 column, 5 μm particle size, 75 μm ID×10 cm length. The column is eluted in five salt steps were used: 0 mM, 40 mM, 80 mM, 125 mM, and 800 mM. The flow rate is approximately 400 mL/min at the column tip. Each of these gradient fractions is run through the 1D column described above and peptides eluted with 2-40% acetonitrile+0.1% formic acid. Peptide identification is performed as described above [0028]. Exemplary base peak chromatograms of the ALS sample and control sample are generated as shown in FIGS. 2 and 3, respectively (“2D LC on ALS Samples” and “2D LC on NC Samples”).

Results: both 1D LC and 2D LC MS/MS indicate that the ALS sample has a noticeably different MS profile from that of the control. SIEVE software was used to analyze and identify corresponding peaks between the two samples and to identify the detected peptide sequences. In the ALS sample, one or more peptides having SEQ ID NOs: 1-42 is detected at greater levels than in the control sample, and/or one or more peptides having SEQ ID NOs: 43-63 is detected at lower levels than in the control sample.

These results demonstrate that use of LC MS/MS can be used to determine levels of biomarker peptides of motor neuron disease.

### Example 2

This example demonstrates a method of identifying post-translational modification of peptide biomarkers of motor neuron disease.

Samples are prepared as described in Example 1 above and run in 1D and 2D LC MS/MS as described above. Post translational modification of the detected peptides is indicated through LC-MS/MS analysis using the Thermo LTQ-Orbitrap XL equipped with Electron Transfer Dissociation (ETD) to detect post-translational modifications to each identified peptide within the sample. Other methods to detect peptide post-translational modifications including purification of phosphopeptide by column chromatography and subsequent antibody detection analysis could also be utilized. In evaluating the test sample, one or more of peptides having SEQ ID NOs:1-63 is found to have post-translational modification differing from those of the control sample.

### Example 3

This example demonstrates a method of diagnosing motor neuron disease.

A sample of CSF is taken from a patient suspected of having ALS. The sample is evaluated using 1D and 2D LC MS/MS as described in Examples 1 and 2, and compared to a control sample from a non-diseased subject. Based on the resulting base peak chromatograms, the sample is found to be elevated in one or more peptides having SEQ ID NOs: 1-42 and/or deficient in one or more peptides having SEQ ID NOs:43-63. Additionally, one or more of the peptides of SEQ ID NOs:1-63 is found to have post-translational modification differing from those of the control sample. This supports a diagnosis of ALS.

### Example 4

This example demonstrates a method of determining a prognosis of motor neuron disease.

Two samples of CSF are taken from a patient clinically diagnosed with ALS at a six-month interval. The samples are evaluated using 1D and 2D LC MS/MS as described in Examples 1 and 2. The resulting base peak chromatograms are compared to each other, and the second sample is found to have increased amounts of one or more peptides having SEQ ID NOs:1-42, and/or decreased amounts of one or more peptides having SEQ ID NOs:43-63. Additionally, one or more of the peptides of SEQ ID NOs:1-63 is found to have increased post-translational modification in the second sample as opposed to the earlier, first sample. Accordingly, a prognosis of advancing ALS is made.

### Example 5

This example demonstrates a method of measuring peptide biomarkers for ALS.

A sample of cerebrospinal fluid (CSF) is taken from a patient recently diagnosed with ALS and a control subject. The control subject groups are healthy, non-diseased controls, subjects with multiple sclerosis, subjects with Alzheimer's disease, subjects with upper motor neuron disease, and subjects with lower motor neuron disease. Each control subject group is analyzed independent of the other control subject groups. The most abundant proteins are removed by affinity chromatography and the samples digested with trypsin and peptides enriched prior to liquid chromatography mass spectrometry (LC-MS/MS). Samples are analyzed on the LC-MS/MS as described in Example 1 for a 1D LC analysis.

Data (Table 2) indicates that ALS samples have specific peptide profile and levels that differentiate ALS from control subject groups. A univariate significance of peptides for each protein between all sample groups is tested by non-parametric Kruskal-Wallis test.

TABLE 2

| PROTEIN | SwissProt # | SEQ ID NO: | p value |
|---|---|---|---|
| Clusterin | P10909 | 10, 55, 56 | 0.0201 |
| Antithrombin III variant | Q7KZ97 | 11-19 | 0.0312 |
| Plasminogen | P00747 | 20, 21 | 0.0106 |
| Coagulation Factor XII | P00748 | 39-42 | 0.0158 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of determining a diagnosis of amyotrophic lateral sclerosis (ALS) comprising:
providing a sample taken from a living human patient;
removing the most abundant proteins by affinity chromatography;
quantifying in the sample the amount of two or more proteins or polypeptides within a panel comprising plasminogen, antithrombin III, coagulation factor XII, and polypeptides of two or more thereof; and
comparing the amount of the two or more proteins or polypeptides with a negative control selected from the group consisting of a sample taken from a non-diseased living human subject and a pre-determined expression profile;
wherein an elevated quantity of the two or more proteins or polypeptides compared to the negative control indicates a diagnosis of ALS.

2. The method of claim 1, wherein one or more proteins or polypeptides comprises a majority of contiguous amino acids of a sequence selected from the group consisting of SEQ ID NOs: 11-21 and 39-42.

3. The method of claim 1, further comprising detecting aberrant post-translational modification of the two or more proteins or polypeptides, wherein the presence of aberrant post-translational modification indicates a diagnosis of ALS.

4. The method of claim 1, wherein the sample is a sample selected from the group consisting of cerebrospinal fluid, blood, and urine.

5. The method of claim 1, wherein the negative control is a sample taken from a non-diseased human subject.

6. A method of determining a prognosis of ALS comprising:
providing a sample taken from a living human patient previously diagnosed with ALS;
removing the most abundant proteins by affinity chromatography;
quantifying in the sample the amount of two or more proteins or polypeptides within a panel comprising plasminogen, antithrombin III, coagulation factor XII, and polypeptides of two or more thereof; and
comparing the amount of two or more proteins or polypeptides with a control selected from the group consisting of a prior sample from the same patient, a sample taken from a non-diseased living human subject, and a pre-determined expression profile;
wherein an elevated quantity of the two or more proteins or polypeptides compared to the control indicates a prognosis of advancing disease and a decreased or unchanged quantity compared to the control indicates a prognosis of non-advancing disease as determined by clinical parameters.

7. The method of claim 6, wherein one or more proteins or polypeptides comprises a majority of contiguous amino acids of a sequence selected from the group consisting of SEQ ID NOs: 11-21 and 39-42.

8. The method of claim 6, further comprising detecting aberrant post-translational modification of the two or more proteins or polypeptides, wherein the presence of aberrant post-translational modification indicates a prognosis of advancing disease.

9. The method of claim 6, wherein the sample is a sample selected from the group consisting of cerebrospinal fluid, blood, and urine.

10. The method of claim 6, wherein the control is a sample taken from a non-diseased human subject.

11. The method of claim 1, wherein a polypeptide consists essentially of an amino acid sequence selected from the group consisting of SEQ ID NOs:11-21 and 39-42, wherein such sequences optionally lack the amino-terminal residue and/or the carboxy-terminal residue.

12. The method of claim 6, wherein a polypeptide consists essentially of an amino acid sequence selected from the group consisting of SEQ ID NOs:11-21 and 39-42 wherein such sequences optionally lack the amino-terminal residue and/or the carboxy-terminal residue.

13. The method of claim 1, wherein the panel comprises (a) plasminogen and/or a polypeptide thereof, (b) antithrombin III and/or a polypeptide thereof, and (c) coagulation factor XII and/or a polypeptide thereof.

14. The method of claim 1, wherein each of the proteins or polypeptides within the panel comprises a majority of contiguous amino acids of a sequence selected from the group consisting of SEQ ID NOs: 11-21 and 39-42.

15. The method of claim 6, wherein the panel comprises (a) plasminogen and/or a polypeptide thereof, (b) antithrombin III and/or a polypeptide thereof, and (c) coagulation factor XII and/or a polypeptide thereof.

16. The method of claim 6, wherein each of the proteins or polypeptides within the panel comprises a majority of contiguous amino acids of a sequence selected from the group consisting of SEQ ID NOs: 11-21 and 39-42.

17. The method of any of claim 1, 2, 6, 7, or 13-16, wherein said polypeptides are the products of enzymatic digestion of said proteins.

18. The method of claim 17, wherein said enzymatic digestion employs trypsin.

19. The method of claim 4 or 9, wherein the sample is cerebrospinal fluid.


SEQUENCE LISTING (63 sequences)

SEQ ID NO: 1 - protein, 11 aa, Homo sapiens
  MISC_FEATURE (1)..(11): Polypeptide from Angiotensin precursor (SwissProt Accession number P01019)
RFMQAVTGWKT

SEQ ID NO: 2 - protein, 14 aa, Homo sapiens
  MISC_FEATURE (1)..(14): Polypeptide from Angiotensin precursor (SwissProt Accession number P01019)
KALQDQLVLVAAKL

SEQ ID NO: 3 - protein, 15 aa, Homo sapiens
  MISC_FEATURE (1)..(15): Polypeptide from Angiotensin precursor (SwissProt Accession number P01019)
RSLDFTELDVAAEKI

SEQ ID NO: 4 - protein, 12 aa, Homo sapiens
  MISC_FEATURE (1)..(12): Polypeptide from Angiotensin precursor (SwissProt Accession number P01019)
RLQAILGVPWKD

SEQ ID NO: 5 - protein, 19 aa, Homo sapiens
  MISC_FEATURE (1)..(19): Polypeptide from Angiotensin precursor (SwissProt Accession number P01019)
KQPFVQGLALYTPVVLPRS

SEQ ID NO: 6 - protein, 20 aa, Homo sapiens
  MISC_FEATURE (1)..(20): Polypeptide from Angiotensin precursor (SwissProt Accession number P01019)
KVLSALQAVQGGLLVAQGRA

SEQ ID NO: 7 - protein, 20 aa, Homo sapiens
  MISC_FEATURE (1)..(20): Polypeptide from Kallikrein 6 (SwissProt Accession number Q92876)
KDSCQGDSGGPLVCGDHLRG

SEQ ID NO: 8 - protein, 22 aa, Homo sapiens
  MISC_FEATURE (1)..(22): Polypeptide from Kallikrein 6 (SwissProt Accession number Q92876)
KTADGDFPDTIQCAYIHLVSRE

SEQ ID NO: 9 - protein, 21 aa, Homo sapiens
  MISC_FEATURE (1)..(21): Polypeptide from Kininogen-1 (SwissProt Accession number P01042)
RETTCSKESNEELTESCETKK

SEQ ID NO: 10 - protein, 13 aa, Homo sapiens
  MISC_FEATURE (1)..(13): Polypeptide from Clusterin (activated by AT-1 receptor binding to ANG-II) (SwissProt Accession number P10909)
RELDESLQVAERL

SEQ ID NO: 11 - protein, 16 aa, Homo sapiens
  MISC_FEATURE (1)..(16): Polypeptide from Antithrombin III (SwissProt Accession number P01008)
KLQPLDFKENAEQSRA

SEQ ID NO: 12 - protein, 20 aa, Homo sapiens
  MISC_FEATURE (1)..(20): Polypeptide from Antithrombin III (SwissProt Accession number P01008)
KATEDEGSEQKIPEATNRRV

SEQ ID NO: 13 - protein, 13 aa, Homo sapiens
  MISC_FEATURE (1)..(13): Polypeptide from Antithrombin III (SwissProt Accession number P01008)
KSKLPGIVAEGRD

SEQ ID NO: 14 - protein, 13 aa, Homo sapiens
  MISC_FEATURE (1)..(13): Polypeptide from Antithrombin III (SwissProt Accession number P01008)
RDDLYVSDAFHKA

SEQ ID NO: 15 - protein, 13 aa, Homo sapiens
  MISC_FEATURE (1)..(13): Polypeptide from Antithrombin III (SwissProt Accession number P01008)
KTSDQIHFFFAKL

SEQ ID NO: 16 - protein, 15 aa, Homo sapiens
  MISC_FEATURE (1)..(15): Polypeptide from Antithrombin III (SwissProt Accession number P01008)
RVAEGTQVLELPFKG

SEQ ID NO: 17 - protein, 14 aa, Homo sapiens
  MISC_FEATURE (1)..(14): Polypeptide from Antithrombin III (SwissProt Accession number P01008)
REVPLNTIIFMGRV

SEQ ID NO: 18 - protein, 23 aa, Homo sapiens
  MISC_FEATURE (1)..(23): Polypeptide from Antithrombin III (SwissProt Accession number P01008)
KNDNDNIFLSPLSISTAFAMTKL

SEQ ID NO: 19 - protein, 27 aa, Homo sapiens
  MISC_FEATURE (1)..(27): Polypeptide from Antithrombin III (SwissProt Accession number P01008)
RITDVIPSEAINELTVLVLVNTIYFKG

SEQ ID NO: 20 - protein, 15 aa, Homo sapiens
  MISC_FEATURE (1)..(15): Polypeptide from Plasminogen (SwissProt Accession number P00747)
KRAPWCHTTNSQVRW

SEQ ID NO: 21 - protein, 23 aa, Homo sapiens
  MISC_FEATURE (1)..(23): Polypeptide from Plasminogen (SwissProt Accession number P00747)
KNYCRNPDGDVGGPWCYTTNPRK

SEQ ID NO: 22 - protein, 31 aa, Homo sapiens
  MISC_FEATURE (1)..(31): Polypeptide from Plasminogen activator, urokinase (SwissProt Accession number Q03405)
RGCATASMCQHAHLGDAFSMNHIDVSCCTKS

SEQ ID NO: 23 - protein, 12 aa, Homo sapiens
  MISC_FEATURE (1)..(12): Polypeptide from Pigment epithelial derived factor precursor (PEDF or Serpin F1) (SwissProt Accession number P36955)
KTVQAVLTVPKL

SEQ ID NO: 24 - protein, 14 aa, Homo sapiens
  MISC_FEATURE (1)..(14): Polypeptide from Pigment epithelial derived factor precursor (PEDF or Serpin F1) (SwissProt Accession number P36955)
KLQSLFDSPDFSKI

SEQ ID NO: 25 - protein, 12 aa, Homo sapiens
  MISC_FEATURE (1)..(12): Polypeptide from Pigment epithelial derived factor precursor (PEDF or Serpin F1) (SwissProt Accession number P36955)
RYGLDSDLSCKI

SEQ ID NO: 26 - protein, 15 aa, Homo sapiens
  MISC_FEATURE (1)..(15): Polypeptide from Pigment epithelial derived factor precursor (PEDF or Serpin F1) (SwissProt Accession number P36955)
RKTSLEDFYLDEERT

SEQ ID NO: 27 - protein, 14 aa, Homo sapiens
  MISC_FEATURE (1)..(14): Polypeptide from Pigment epithelial derived factor precursor (PEDF or Serpin F1) (SwissProt Accession number P36955)
KTSLEDFYLDEERT

SEQ ID NO: 28 - protein, 19 aa, Homo sapiens
  MISC_FEATURE (1)..(19): Polypeptide from Pigment epithelial derived factor precursor (PEDF or Serpin F1) (SwissProt Accession number P36955)
RALYYDLISSPDIHGTYKE

SEQ ID NO: 29 - protein, 17 aa, Homo sapiens
  MISC_FEATURE (1)..(17): Polypeptide from Pigment epithelial derived factor precursor (PEDF or Serpin F1) (SwissProt Accession number P36955)
RLDLQEINNWVQAQMKG

SEQ ID NO: 30 - protein, 19 aa, Homo sapiens
  MISC_FEATURE (1)..(19): Polypeptide from Pigment epithelial derived factor precursor (PEDF or Serpin F1) (SwissProt Accession number P36955)
KEIPDEISILLLGVAHFKG

SEQ ID NO: 31 - protein, 12 aa, Homo sapiens
  MISC_FEATURE (1)..(12): Polypeptide from Vitamin D-binding protein precursor (SwissProt Accession number P02774)
RTHLPEVKLSKV

SEQ ID NO: 32 - protein, 18 aa, Homo sapiens
  MISC_FEATURE (1)..(18): Polypeptide from Vitamin D-binding protein precursor (SwissProt Accession number P02774)
KELPEHTVKLCDNLSTKN

SEQ ID NO: 33 - protein, 15 aa, Homo sapiens
  MISC_FEATURE (1)..(15): Polypeptide from Vitamin D-binding protein variant (SwissProt Accession number Q53F31)
KAKLPEATPTELAKL

SEQ ID NO: 34 - protein, 18 aa, Homo sapiens
  MISC_FEATURE (1)..(18): Polypeptide from Insulin-like growth factor binding protein 7 (IGFBP-7) (SwissProt Accession number Q16270)
RTELLPGDRDNLAIQTRG

SEQ ID NO: 35 - protein, 24 aa, Homo sapiens
  MISC_FEATURE (1)..(24): Polypeptide from Insulin-like growth factor binding protein 7 (IGFBP-7) (SwissProt Accession number Q16270)
KEDAGEYECHASNSQGQASASAKI

SEQ ID NO: 36 - protein, 17 aa, Homo sapiens
  MISC_FEATURE (1)..(17): Polypeptide from Insulin-like growth factor binding protein 6 (IGFBP-6) (SwissProt Accession number P24592)
RHLDSVLQQLQTEVYRG

SEQ ID NO: 37 - protein, 17 aa, Homo sapiens
  MISC_FEATURE (1)..(17): Polypeptide from Insulin-like growth factor binding protein 6 (IGFBP-6) (SwissProt Accession number P24592)
RCLPARAPAVAEENPKE

SEQ ID NO: 38 - protein, 24 aa, Homo sapiens
  MISC_FEATURE (1)..(24): Polypeptide from Insulin-like growth factor binding protein 6 (IGFBP-6) (SwissProt Accession number P24592)
REGQECGVYTPNCAPGLQCHPPKD

SEQ ID NO: 39 - protein, 20 aa, Homo sapiens
  MISC_FEATURE (1)..(20): Polypeptide from Coagulation Factor XII (SwissProt Accession number P00748)
RTTLSGAPCQPWASEATYRN

SEQ ID NO: 40 - protein, 20 aa, Homo sapiens
  MISC_FEATURE (1)..(20): Polypeptide from Coagulation Factor XII (SwissProt Accession number P00748)
RLHEAFSPVSYQHDLALLRL

SEQ ID NO: 41 - protein, 19 aa, Homo sapiens
  MISC_FEATURE (1)..(19): Polypeptide from Coagulation Factor XII (SwissProt Accession number P00748)
RNKPGVYTDVAYYLAWIRE

SEQ ID NO: 42 - protein, 20 aa, Homo sapiens
  MISC_FEATURE (1)..(20): Polypeptide from Coagulation Factor XII (SwissProt Accession number P00748)
RLCHCPVGYTGPFCDVDTKA

SEQ ID NO: 43 - protein, 21 aa, Homo sapiens
  MISC_FEATURE (1)..(21): Polypeptide from Angiotensin precursor (SwissProt Accession number P01019)
KANAGKPKDPTFIPAPIQAKT

SEQ ID NO: 44 - protein, 20 aa, Homo sapiens
  MISC_FEATURE (1)..(20): Polypeptide from Kallikrein 6 (SwissProt Accession number Q92876)
RLARPAKLSELIQPLPLERD

SEQ ID NO: 45 - protein, 14 aa, Homo sapiens
  MISC_FEATURE (1)..(14): Polypeptide from Kallikrein 6 (SwissProt Accession number Q92876)
KLSELIQPLPLERD

SEQ ID NO: 46 - protein, 15 aa, Homo sapiens
  MISC_FEATURE (1)..(15): Polypeptide from Kallikrein 6 (SwissProt Accession number Q92876)
RGLVSWGNIPCGSKE

SEQ ID NO: 47 - protein, 15 aa, Homo sapiens
  MISC_FEATURE (1)..(15): Polypeptide from Kallikrein 6 (SwissProt Accession number Q92876)
RQRESSQEQSSVVRA

SEQ ID NO: 48 - protein, 23 aa, Homo sapiens
  MISC_FEATURE (1)..(23): Polypeptide from Kallikrein 6 (SwissProt Accession number Q92876)
KYGKDSCQGDSGGPLVCGDHLRG

SEQ ID NO: 49 - protein, 22 aa, Homo sapiens
  MISC_FEATURE (1)..(22): Polypeptide from Kallikrein 6 (SwissProt Accession number Q92876)
RAVIHPDYDAASHDQDIMLLRL

SEQ ID NO: 50 - protein, 11 aa, Homo sapiens
  MISC_FEATURE (1)..(11): Polypeptide from Kininogen-1 (SwissProt Accession number P01042)
KRPPGFSPFRS

SEQ ID NO: 51 - protein, 15 aa, Homo sapiens
  MISC_FEATURE (1)..(15): Polypeptide from Kininogen-1 (SwissProt Accession number P01042)
RIGEIKEETTSHLRS

SEQ ID NO: 52 - protein, 22 aa, Homo sapiens
  MISC_FEATURE (1)..(22): Polypeptide from Kininogen-1 (SwissProt Accession number P01042)
RKLGQSLDCNAEVYVVPWEKKI

SEQ ID NO: 53 - protein, 16 aa, Homo sapiens
  MISC_FEATURE (1)..(16): Polypeptide from Kininogen-1 (SwissProt Accession number P01042)
KGRPPKAGAEPASERE

SEQ ID NO: 54 - protein, 15 aa, Homo sapiens
  MISC_FEATURE (1)..(15): Polypeptide from Kininogen-1 (SwissProt Accession number P01042)
KAATGECTATVGKRS

SEQ ID NO: 55 - protein, 13 aa, Homo sapiens
  MISC_FEATURE (1)..(13): Polypeptide from Clusterin (activated by AT-1 receptor binding to ANG-II) (SwissProt Accession number P10909)
RKTLLSNLEEAKK

SEQ ID NO: 56 - protein, 16 aa, Homo sapiens
  MISC_FEATURE (1)..(16): Polypeptide from Clusterin (activated by AT-1 receptor binding to ANG-II) (SwissProt Accession number P10909)
KYVNKEIQNAVNGVKQ

SEQ ID NO: 57 - protein, 14 aa, Homo sapiens
  MISC_FEATURE (1)..(14): Polypeptide from Pigment epithelial derived factor precursor (PEDF or Serpin F1) (SwissProt Accession number P36955)
RDTDTGALLFIGKI

SEQ ID NO: 58 - protein, 13 aa, Homo sapiens
  MISC_FEATURE (1)..(13): Polypeptide from Vitamin D-binding protein precursor (SwissProt Accession number P02774)
KLPDATPTELAKL

SEQ ID NO: 59 - protein, 27 aa, Homo sapiens
  MISC_FEATURE (1)..(27): Polypeptide from Vitamin D-binding protein precursor (SwissProt Accession number P02774)
KELSSFIDKGQELCADYSENTFTEYKK

SEQ ID NO: 60 - protein, 26 aa, Homo sapiens
  MISC_FEATURE (1)..(26): Polypeptide from Vitamin D-binding protein precursor (SwissProt Accession number P02774)
KSLGECCDVEDSTTCFNAKGPLLKKE

SEQ ID NO: 61 - protein, 21 aa, Homo sapiens
  MISC_FEATURE (1)..(21): Polypeptide from Insulin-like growth factor binding protein 7 (IGFBP-7) (SwissProt Accession number Q16270)
RGGPEKHEVTGWVLVSPLSKE

SEQ ID NO: 62 - protein, 21 aa, Homo sapiens
  MISC_FEATURE (1)..(21): Polypeptide from Insulin-like growth factor binding protein 7 (IGFBP-7) (SwissProt Accession number Q16270)
RGKAGAAAGGPGVSGVCVCKS

SEQ ID NO: 63 - protein, 21 aa, Homo sapiens
  MISC_FEATURE (1)..(21): Polypeptide from Insulin-like growth factor binding protein 7 (IGFBP-7) (SwissProt Accession number Q16270)
RITVVDALHEIPVKKGEGAEL